(19) United States
(12) Plant Patent Application Publication
(10) Pub. No.: US 2005/0216988 P1
(43) Pub. Date: Sep. 29, 2005

(54) CALIBRACHOA PLANT NAMED 'SUNBELBUSTA'

(50) PETUNIA HYBRIDA; PETUNIA

(76) Inventors: Yasuyuki Murakami, Gamo-gun (JP); Takeshi Kanaya, Omihachiman (JP)

Correspondence Address: CHRISTIE, PARKER & HALE, LLP, PO BOX 7068, PASADENA, CA, 91109-7068, US

(21) Appl. No.: 10/810,496

(22) Filed: Mar. 25, 2004

(51) Int. Cl. A01H 5/00

(52) U.S. Cl. PLT/263

(57) ABSTRACT

Disclosed herein is a new and distinct variety of Calibrachoa plant having an outwardly spreading growth habit. The Calibrachoa plant has abundant branching, and a great profusion of blooms, the whole plant remaining in bloom for a considerable period of time. The flowers are single and very small, the petals having a vivid purple color with deep purple vein. The inside color of the corolla throat is vivid yellow with brilliant yellow-green and the outside of the corolla tube is strong purple with dark reddish-purple. The plant exhibits high resistance to heat, cold, rain and disease.

### BOTANICAL/COMMERCIAL CLASSIFICATION

Calibrachoa sp./Calibrachoa Plant

### VARIETAL DENOMINATION

cv. ‘Sunbelbusta’

### BACKGROUND OF THE VARIETY

The present invention relates to a new and distinct variety of Calibrachoa plant, botanically known as Calibrachoa sp., which originated from crossing of a Calibrachoa hybrid variety called ‘CHBS05’ as the female parent and ‘CHBS07’ as the male parent.

The female parent ‘CHBS05’ (unpatented) used in the crossing of ‘Sunbelbusta’ is a strain of our breeding lines, having an erect growth habit. It has small single flowers, the petals having a vivid red-purple color.

The male parent ‘CHBS07’ (unpatented) used in the crossing of ‘Sunbelbusta’ is a strain of our breeding lines, having a decumbent growth habit. it has small single flowers, the petals having violet color.

In July 1998, crossing of ‘CHBS05’ as the female parent and ‘CHBS07’ as the male parent was conducted at Yokaichi-shi, Shiga-ken, Japan. In October 1998, seedlings were obtained from that crossing. These seedlings were grown in pots in glasshouses and were evaluated. One seedling was selected in view of its growth habit, flower size and color in October 1999. That seedling was propagated by cutting and a trial was carried out by flower potting and bedding from April to October 2000. The botanical characteristics of that plant were then examined, using similar varieties ‘Sunbelribu’ (unpatented) and ‘Sunbelkubu’ (U.S. Plant Pat. No. 10,279) for comparison. As a result, it was concluded that this Calibrachoa plant is distinguishable from any other variety whose existence is known to us, and is uniform and stable in its characteristics. The new variety of Calibrachoa plant was named ‘Sunbelbusta’.

In the following description, the color-coding is in accordance with the Horticultural Colour Chart of The Royal Horticultural Society, London, England (R.H.S. Colour Chart).

### SUMMARY OF THE VARIETY

This new variety is unlike any Calibrachoa commercially available as evidenced by the following unique combinations of characteristics.

1. Outwardly spreading growth habit with abundant branching.

2. Great profusion of blooms with the entire plant remaining in bloom for a considerable period of time.

3. The flowers are single and very small. The apex shape of petal is acute. The petal color is vivid purple (near R.H.S. N81A) with deep purple (near R.H.S.83A) vein.

4. The plant has a high resistance to rain, cold, heat and disease.

The new variety ‘Sunbelbusta’ differs from the similar variety ‘Sunbelribu’ in the following points.

1. The spreading area of ‘Sunbelbusta’ is larger than that of ‘Sunbelribu’.

2. The internode length of ‘Sunbelbusta’ is shorter than that of ‘Sunbelribu’.

3. The flower diameter of ‘Sunbelbusta’ is larger than that of ‘Sunbelribu’.

4. The petal color of ‘Sunbelbusta’ is vivid purple (near R.H.S.N81A) with deep purple (near R.H.S.83A) vein, while that of ‘Sunbelribu’ is vivid purple (near R.H.S.N87A).

5. Inside color of the corolla throat of ‘Sunbelbusta’ is vivid yellow (near R.H.S. 7B) with brilliant yellowish green (near R.H.S. N199A) vein. That of ‘Sunbelribu’ is vivid greenish-yellow (near R.H.S.151C).

6. Outside color of the corolla tube of ‘Sunbelbusta’ is strong purple (R.H.S.83D) with dark reddish-purple (near R.H.S.79A). That of ‘Sunbelribu’ is light yellow-green (near R.H.S.1C).

The new variety ‘Sunbelbusta’ differs from the similar variety ‘Sunbelkubu’ in the following points.

1. The growth habit of ‘Sunbelbusta’ is outwardly spreading, while that of ‘Sunbelkubu’ is decumbent.

2. The plant height of ‘Sunbelbusta’ is higher than that of ‘Sunbelkubu’.

3. The leaf length of ‘Sunbelbusta’ is longer than that of ‘Sunbelkubu’.

4. The petal color of ‘Sunbelbusta’ is vivid purple (near R.H.S.N81A) with deep purple (near R.H.S.83A), while that of ‘Sunbelkubu’ is vivid purple (R.H.S.N81A).

5. Inside color of the corolla throat of ‘Sunbelbusta’ is vivid yellow (near R.H.S. 7B) with brilliant yellowish green (near R.H.S. N199A) venation, while that of ‘Sunbelkubu’ is brilliant greenish yellow (near R.H.S.5C).

6. Outside color of the corolla tube of ‘Sunbelbusta’ is strong purple (R.H.S.83D) with dark reddish purple (R.H.S.79A), while that of ‘Sunbelkubu’ is pale yellow green (R.H.S.1D).

7. The apex shape of the petal of ‘Sunbelbusta’ is acute, while that of ‘Sunbelkubu’ is obtuse.

This new variety of Calibrachoa plant ‘Sunbelbusta’ was asexually reproduced by the use of cuttings at Yokaichi-shi, Shiga-ken, Japan, and homogeneity and stability thereof were confirmed. The instant plant retains its distinctive characteristics and produces true to type in successive generations.

### BRIEF DESCRIPTION OF THE PHOTOGRAPHS

The depicted plants have been reproduced by the use of cuttings and were photographed during April 2003 while growing outdoors in 20 cm wall pots at an age of approximately 4 months at Yokaichi-shi, Shiga-ken, Japan.

FIG. 1 is a photograph of a typical plant of the new variety of Calibrachoa plant ‘Sunbelbusta’ while growing in a pot.

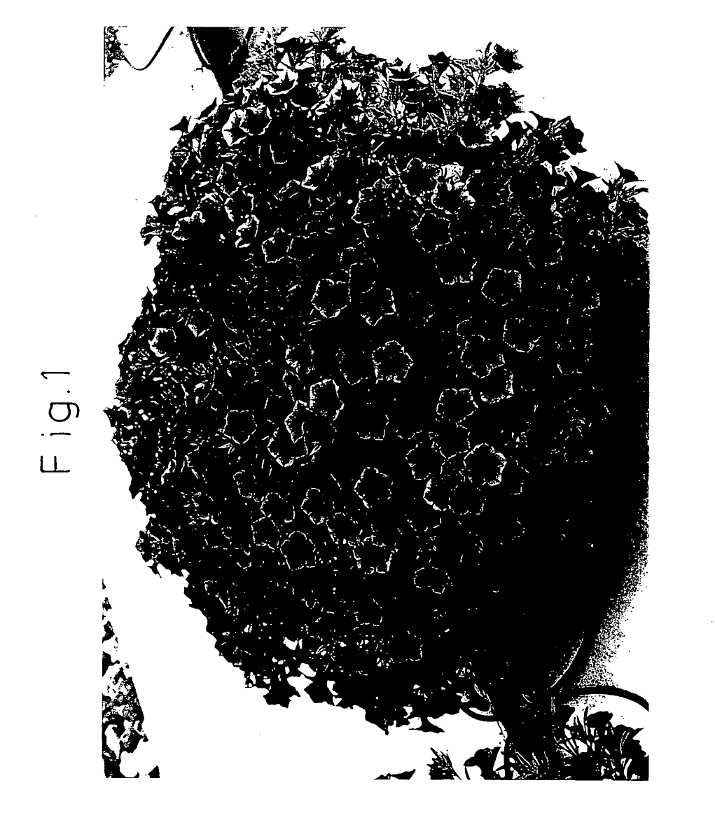

FIG. 2 is a close view of flowers and leaves of the new variety of Calibrachoa plant ‘Sunbelbusta’.

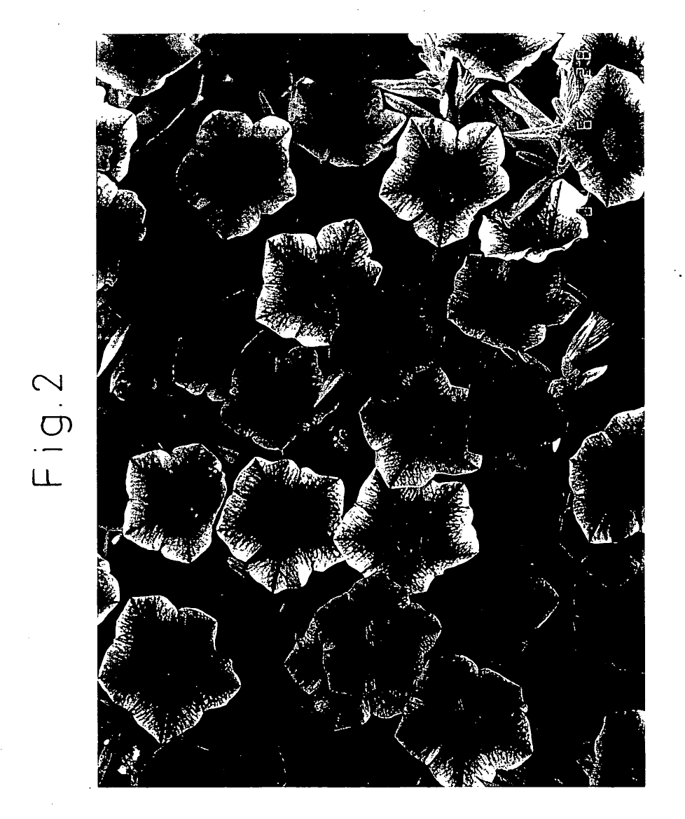

### DESCRIPTION OF THE VARIETY

The botanical characteristics of the new and distinct variety of Calibrachoa plant named ‘Sunbelbusta’ are as follows when observed during September at Yokaichi-shi, Shiga-ken, Japan, at an age of approximately 9 months.
Plant:
   Growth habit.—Outwardly spreading.
   Plant height.—Approximately 23.8 cm.
   Spreading area of plant.—Approximately 49.0 cm.
   Blooming period.—April to late October in the southern Kanto area, Japan. The plant shape does not change throughout this period.
Stem:
   Length.—approximately 30.0 cm.
   Thickness.—Approximately 1.8 mm.
   Pubescence.—Normal.
   Branching.—Abundant branching, especially secondary branches.
   Internode length.—Approximately 0.9 cm.
   Color.—Near R.H.S. 165A (grayish yellow brown).
Leaf:
   Whole shape.—Lanceolate. The apex shape is acute, and the base shape is attenuate.
   Length.—Approximately 4.4 cm.
   Width.—Approximately 1.0 cm.
   Color.—Upper side color is near R.H.S. 137C (moderate yellow green). Lower side color is near R.H.S. 146C (moderate yellow green).
   Thickness.—Approximately 0.5 mm.
   Pubescence.—Normal.
Buds:
   Shape.—Cylindrical
   Length.—Approximately 2.0 cm.
   Diameter.—Approximately 4.0 mm.
   Color.—towards apex, near R.H.S. N81B; Base — near R.H.S. 151D; Venation — near R.H.S. 79A.
Flower:
   Petals:
   Width.—Approximately 1.3 cm.
   Length from throat.—Approximately 1.4 cm.
   Shape.—Broadly obtrullate.
   Margin.—Entire.
   Texture.—Smooth.
   Color.—Lower surface, near R.H.S. N81B with near R.H.S. 79A venation.
   Facing direction.—Horizontal.
   Type.—single.
   Shape.—Funnel-shaped, with five-fissures.
   Shape of petal tip.—Acute.
   Lobation.—Medium
   Waviness of petal.—Weak.
   Diameter.—Approximately 3.5 cm.
   Depth.—approximately 2.7 cm.
   Tube length.—Approximately 1.8 cm.
   Throat diameter.—Distal end — Approximately 0.8 cm.
   Tube diameter.—Proximal end — Approximately 2.0 mm.
   Color.—Petal; near R.H.S. N81A (vivid purple) with near R.H.S. 83A (deep purple) vein. Bottom color of the corolla throat; near R.H.S. 7B (vivid yellow) with near R.H.S. N199A (brilliant yellowish green) vein. Outside color of the corolla tube; near R.H.S. 83D (strong purple) with near R.H.S.79A (dark reddish purple) vein. Upper color of the corolla throat — near R.H.S. 83A.
   Reproductive organs.—1 normal pistil and 5 normal stamens. The stigma is club-shaped and near R.H.S.144C (strong yellow green) in coloration. The style is approximately 6 mm in length and near R.H.S. 144D (light yellow green) in coloration. The ovary is near R.H.S. 165A (grayish reddish purple) in coloration. The stamens commonly are of variable length from approximately 5.7 to 9.1 mm. Pollen is formed in a quantity that is typical of Calibrachoa sp. and is near R.H.S.9B (vivid yellow) in coloration.
   Fertility.—Fertile, but self-incompatible.
   Peduncle.—Approximately 0.7 mm in diameter and approximately 1.8 cm in length. The texture is smooth. Color is near R.H.S. 144B.
   Seeds.—Near R.H.S. N186A (strong red) in coloration, approximately 0.6 mm in diameter, and generally round. The quantity is typical of Calibrachoa sp.
Sepals:
   Shape.—Narrow elliptic.
   Apex shape.—Obtuse.
   Base.—Fused.
   Margin.—Entire.
   Length.—Approximately 1.4 cm.
   Width.—Approximately 3.0 mm.
   Color.—Upper and lower surfaces near R.H.S. 137C.
   Calyx.—Narrow. 5 sepals fused at the base.
Physiological and ecological characteristics: High resistance to cold, heat, rain and disease such as Powdery mildew. The resistance to heat and rain is very strong.
Blooming: This new variety of Calibrachoa plant is most suitable for flower bedding and potting, particularly in hanging pots or planters. Pinching of old blossoms will enhance the formation of new blossoms.

1. A new and distinct variety of Calibrachoa plant named ‘Sunbelbusta’, substantially as herein illustrated and described.
